(19) United States
(12) Patent Application Publication
(10) Pub. No.: US 2007/0083219 A1
(43) Pub. Date: Apr. 12, 2007

(54) Embolic coil introducer sheath locking mechanisms

(76) Inventors: Marcia S. Buiser, Watertown, MA (US); Christopher J. Elliott, Hopkinton, MA (US); Ashley Seehusen, Newton, MA (US); Christopher Nardone, N. Chelmsford, MA (US)

Correspondence Address: FISH & RICHARDSON PC, P.O. BOX 1022, MINNEAPOLIS, MN, 55440-1022, US

(21) Appl. No.: 11/248,493

(22) Filed: Oct. 12, 2005

(51) Int. Cl. A61B 17/08

(52) U.S. Cl. 606/157

(57) ABSTRACT

Embolic coil introducer sheath locking mechanisms, as well as related components, systems and methods are disclosed.

### TECHNICAL FIELD

The invention relates to embolic coil introducer sheath locking mechanisms, as well as related components, systems and methods.

### BACKGROUND

Embolic coils can be used to occlude vessels in a variety of medical applications. In many instances, an embolic coil is contained in an introducer sheath. The introducer sheath containing the embolic coil is disposed within a body lumen (e.g., an artery) of a subject (e.g., a human), and the embolic coil is pushed out the distal end of the introducer sheath, using, for example, a pusher wire.

### SUMMARY

The invention relates to embolic coil introducer sheath locking mechanisms, as well as related components, systems and methods.

In a first aspect, systems are provided that include an embolic coil introducer sheath and a locking mechanism. The locking mechanism has a first position in which the locking mechanism applies pressure to the embolic coil introducer sheath and has a second position in which the locking mechanism does not apply pressure to the embolic coil introducer sheath. The locking mechanism is removable from the embolic coil introducer sheath.

In a second aspect, systems are provided that include an embolic coil introducer sheath and a locking mechanism having first and second positions. The locking mechanism applies pressure to the embolic coil introducer sheath when the locking mechanism is in the first position, and does not apply pressure to the embolic coil introducer sheath when the locking mechanism is in the second position. The locking mechanism can be moved from the second position to the first position.

In a third aspect, systems are provided that include an embolic coil introducer sheath and a locking mechanism having first and second positions. The locking mechanism applies pressure to the embolic coil introducer sheath when the locking mechanism is in the first position, and does not apply pressure to the embolic coil introducer sheath when the locking mechanism is in the second position. The locking mechanism can be repeatedly moved between the first and second positions.

In another aspect, systems are provided including an embolic coil introducer sheath and a clamp having first and second positions. The clamp applies pressure to the embolic coil introducer sheath when the clamp is in the first position, and the clamp does not apply pressure to the embolic coil introducer sheath when the clamp is in the second position.

In still another aspect, systems are provided including a resilient material having a bore, an embolic coil introducer sheath disposed in the bore of the resilient material, and a member configured to translate an axial force on the member into a radial compressive force on the embolic coil introducer sheath.

In yet another aspect, systems are provided having an embolic coil introducer sheath and a Touhy-Borst valve configured to apply pressure against the embolic coil introducer sheath.

In another aspect, systems are provided having an embolic coil introducer sheath, an embolic coil pusher wire, and a wedge configured to fit between the embolic coil introducer sheath and the embolic coil pusher wire.

In an additional aspect, systems are provided having an embolic coil introducer sheath and a sleeve comprising a polymer material and having at least one perforation. The sleeve is supported by the embolic coil introducer sheath.

In a further aspect, perforated embolic coil introducer sheaths are provided.

In still another aspect, systems are provided having an embolic coil pusher wire and an embolic coil introducer sheath having at least one perforation. The perforated embolic coil introducer sheath includes a locking portion that restricts longitudinal movement of the embolic coil pusher wire in the perforated embolic coil introducer sheath when the locking portion is in a first position and does not restrict longitudinal movement of the embolic coil pusher wire in the perforated embolic coil introducer sheath when the locking portion is in a second position.

Embodiments can include one or more of the following advantages.

In some embodiments, the locking mechanism can allow for easier control and/or use.

In some embodiments, the locking mechanisms may provide a relatively secure and/or reliable lock on the delivery wire.

In some embodiments, the locking mechanism may be repeatedly moved between a position in which the locking mechanism locks an embolic coil and a position in which the locking mechanism does not lock an embolic coil. This can, for example, allow for greater flexibility in delivering an embolic coil. As an example, the embolic coil can be locked in a first position, unlocked, moved to a second position and then relocked. In some embodiments, the locking mechanism retains substantially the same (e.g., full) degree of locking power of the locking mechanism (e.g., the locking power is substantially unchanged) upon being unlocked and subsequently relocked.

In some embodiments, the locking mechanism may be capable of being unlocked and removed and then repositioned and relocked.

In some embodiments, the locking mechanism may be capable of one-handed operation, that is to say, may be capable of being removed and/or replaced by an operator using only one hand.

Other features and advantages are apparent from the description, drawings and claims.

### DESCRIPTION OF DRAWINGS

FIG. 1A is a partial cross-sectional view of an embodiment of an embolic coil introducer system.

FIG. 1B is a partial cross-sectional view of an embodiment of an embolic coil introducer system in an unlocked position.

FIG. 2A is a perspective view of an embodiment of an embolic coil introducer system.

FIG. 2B is a side view of an embodiment of an embolic coil introducer system.

FIG. 2C is a side view of an embodiment of an embolic coil introducer system in an unlocked position.

FIG. 3 is a side view of an embodiment of an embolic coil introducer system.

FIG. 4 is a perspective view of an embolic coil introducer system.

FIG. 5 is a cross-sectional view of an embolic coil introducer system.

FIG. 6 is a partial cross-sectional view of an embodiment of an embolic coil introducer system.

FIG. 7A is a partial cross-sectional view of an embodiment of an embolic coil introducer system.

FIG. 7 B is partial cross-sectional view of an embodiment of an embolic coil introducer system in an unlocked position.

Like reference symbols in the various drawings indicate like elements.

### DETAILED DESCRIPTION

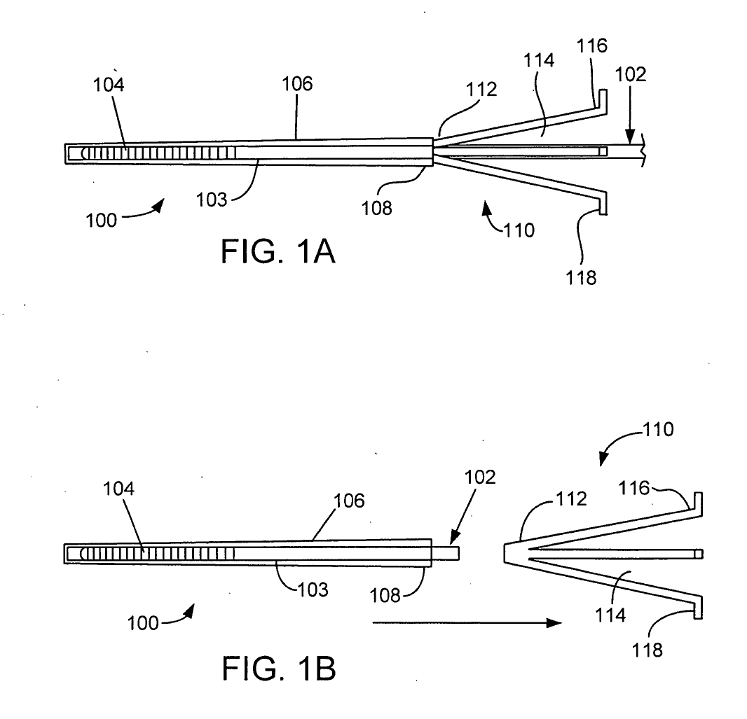

FIGS. 1A and 1B show a distal portion of an embolic coil assembly 100, including a distal portion 103 of a delivery wire 102 and an embolic coil 104, which is contained within an introducer sheath 106. A wedge-shaped locking mechanism 110 extends around the delivery wire 102 and locks the delivery wire 102, and thus the embolic coil assembly 100, in place relative to the introducer sheath 106, as illustrated in FIG. 1A. The wedge-shaped locking mechanism 110 has a generally cylindrical shape and is tapered at a distal end 112 to form a wedge when inserted into the proximal end 108 of the introducer sheath 106. This wedge fit results in force being directed radially inward, which compresses the introducer sheath 106 against the delivery wire 102 and locks the delivery wire 102 in place. The wedge-shaped locking mechanism 110 includes slots 114 that can aid in forming a wedge fit with the introducer sheath 106. The wedge-shaped locking mechanism 110 is removed by moving it proximally along the delivery wire 102 such that the distal end 112 of wedge-shaped locking mechanism 110 disengages from the proximal end 108 of the introducer sheath 106, thus eliminating the compression of the introducer sheath 106 against the delivery wire 102. Extensions 118 are located at a proximal end 116 of the wedge-shaped locking mechanism 110 and extend outward radially so that the locking mechanism 110 can be gripped and pulled out of the introducer sheath 106. The wedge-shaped locking mechanism 110 may then either be left on the delivery wire 102 or may be slid back along the delivery wire 102 and removed. If removed entirely, the wedge-shaped locking mechanism 110 can be placed over a proximal end of the delivery wire 102 and slid distally to the introducer sheath 106, where it can be used to relock the embolic coil assembly 100 in position within the introducer sheath 106.

In general, wedge-shaped locking mechanism 110 may be formed of a deformable material that can deform to fit the profile of the proximal end 108 of the introducer sheath 106 (see discussion above). Exemplary types of deformable materials include rubbers, synthetic rubbers, thermoplastic elastomers and foams. Examples of deformable materials include polyurethanes, Santoprenes™, low density polyethylene, silicone, polyisoprene, vinyl, polyvinyl chloride, ethylene vinyl acetate, and polypropylene. The wedge-shaped locking mechanism may be in the form of a deformable structure, which itself may be formed of a deformable material or a material that is not deformable. Exemplary deformable structures include braided, knit or woven metallic or polymeric structures. Combinations of materials can be used.

Embodiments of a wedge-shaped locking mechanism may include more than one slot (e.g., two or more slots, three or more slots, four or more slots, five or more slots). In embodiments that include more than one slot, different slots may extend the same length or different lengths. For example, one slot may extend the entire length of the wedge-shaped locking mechanism, enabling the locking mechanism to be opened and slipped off of the delivery wire and/or introducer sheath without necessitating sliding it off of the proximal end of the delivery wire. Similarly, such an embodiment may be reinstalled over the delivery wire without need to slide it the length of the delivery wire.

In certain embodiments, a wedge-shaped locking mechanism may be configured to lock the delivery wire in the introducer sheath without having any slots. For example, in such embodiments, the introducer sheath may be tapered or flared at its proximal end to further enable the formation of a snug fit with the wedge-shaped locking mechanism.

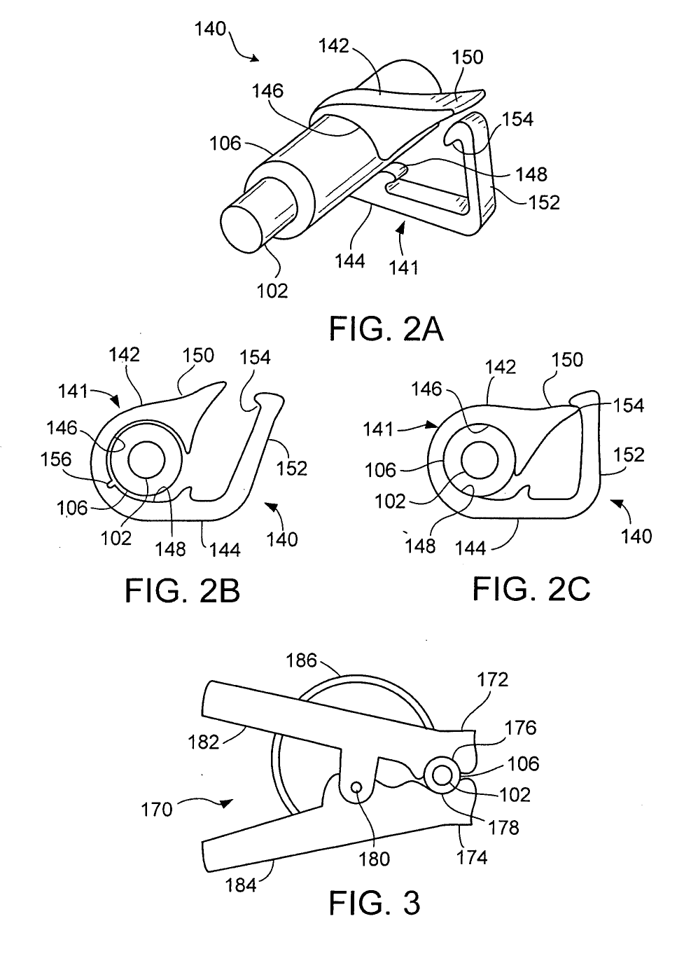

FIGS. 2A and 2B show an embodiment of an embolic coil introducer system 140 having a clamp 141 that includes a first extension 142 and a second extension 144 that combine to partially surround and apply pressure to introducer sheath 106, locking it against delivery wire 102. First and second extensions 142 and 144 include sheath-contacting surfaces 146 and 148, respectively, that contact the introducer sheath 106 when the clamp 141 is in a locked position. A locking extension 150 extends away from the first extension 142 in a direction substantially at a right angle to a longitudinal axis of the introducer sheath 106, and a locking arm 152 with a ratchet 154, capable of engaging the locking extension 150, extends away from the second extension. The first and second extensions 142, 144 are biased away from each other such that the sheath-contacting surfaces 146, 148 do not contact the introducer sheath when the locking extension 150 and the ratchet 154 are not engaged. Engaging of the locking extension 150 and the ratchet 154 biases the first and second extensions 142, 144 towards each other and results in pressure being applied to the introducer sheath 106 by sheath-contacting surfaces 146, 148, such that the delivery wire 102 is restrained from movement within the introducer sheath 106. The clamp 141 is shown in an unlocked position in FIG. 2A and in a locked position in FIG. 2B. The locking mechanism illustrated in FIG. 2B includes an optional cutout 156, which can act to aid in the separation of the first and second extensions 142 and 144, easing the unlocking of the clamp 141. Such a cutout can also serve as a hinge point to permit the clamp 141 to be opened sufficiently to be slid off of the introducer sheath 106 without the need for sliding it to the proximal end of the delivery wire 102.

In general, the clamp 141 can be formed of a material capable of flexing to at least some degree to enable the clamp to be clamped around the sheath and to permit clamp to be unclamped and removed. Exemplary materials from which clamp 141 can be formed include plastics and metals. Examples of plastics include acrylonitrile butadiene styrene copolymer (ABS), polyvinyl chloride (PVC), low density polyethylene (LDPE), high density polyethylene, polypropylene, polyethylene, high density polypropylene, polyethylene terephthalate, nylon, silicone, vinyl, acrylic, polycarbonate and polyacetal (e.g., Delrin®). Examples of metals include stainless steel, Nitinol (NiTi), aluminum, titanium, cobalt-chromium alloys, and Elgiloy®.

Another embodiment of an embolic coil introducer system is illustrated in FIG. 3. Locking mechanism 170 has first and second extensions 172 and 174 having sheath-contacting surfaces 176 and 178, respectively. First and second extensions 172 and 174 are joined by a hinge 180. First and second extensions 172 and 174 have rear portions 182 and 184 located on a side opposite the hinge 180 from the sheath-contacting surfaces 176 and 178. In this embodiment, the sheath contacting surfaces 176 and 178 are biased towards each other by a spring 186 connected to rear portion 184 of first extension 174 and to first extension 172 in such a way as to bias first extension 172 towards second extension 174.

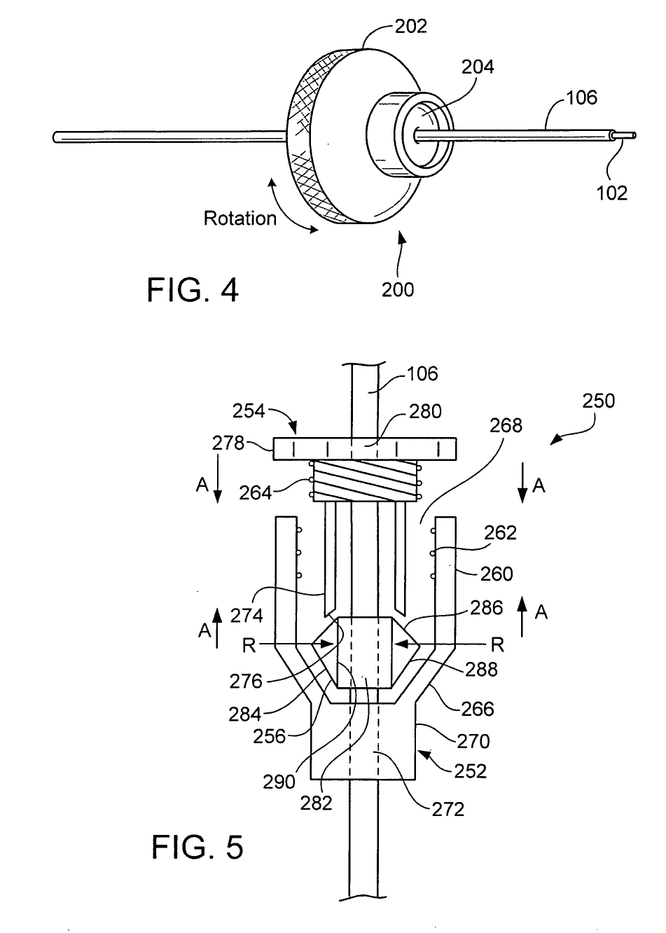

In some embodiments, the locking mechanism is a device that translates axial force into radial compressive force. An example of such a locking mechanism is a Touhy-Borst adapter 200 (FIG. 4). Rotation of a rotating collar 202 results in compression of an O-ring 204 around the introducer sheath 106, locking it against a delivery wire contained within.

FIG. 5 illustrates an embodiment in which a locking mechanism 250 translates axial force (indicated by arrows A) into radial compressive force (indicated by arrows R). In this embodiment, a receiving member 252 is adapted to receive a cap 254 to compress a compressible member 256 radially against the introducer sheath 106, locking a delivery wire (not illustrated) within the sheath 106. The receiving member 252 includes a socket 260 having threads 262 on the interior for receiving a threaded portion 264 of the cap 254. A tapered portion 266 extends away from the open end 268 of the socket 260 and tapers from a larger diameter at the socket 260 to a smaller diameter at an end away from the socket 260. The tapered portion 266 ends at a tubular portion 270, which has a central bore 272 for receiving the introducer sheath 106. The cap 254 has a collar 278 for rotating the cap 254 to screw threaded portion 264 into the threaded socket 260 of the receiving member 252. The cap 254 has a central bore 280 for receiving the introducer sheath 106. The cap 254 further includes a generally tubular extension 274 which ends with tapered surfaces 276 angled inwardly. The compressible member 256 has a central bore 282 for receiving the introducer sheath. The compressible member 256 further includes a side wall 284 having a dual-tapered design, with an upper portion 286 tapering outwardly from the top to about the middle of the compressible member 256 and a lower portion 288 tapering outwardly from the bottom to about the middle of the compressible member 256.

In operation, the introducer sheath 106 may be inserted through the central bores 280, 282 and 272 of the cap 254, the compressible member 256 and the receiving member 252 separately, or the locking mechanism 250 may be threaded together prior to inserting the introducer sheath through the central bores 280, 282 and 272, generally provided that the compressible member 256 remains in an un-compressed state. Once the locking mechanism 250 is in the desired location, the threaded portion 264 of the cap 254 is threaded into the threaded socket 260 of the receiving member 252 and screwed down, resulting in axial translation of the generally tubular extension 274 toward the tapered portion 266 of the receiving member 252. The tapered surfaces of the tapered portion 266 and of the tubular extension 274 interact with the tapered portions 286 and 288 of the compressible member 256 to force an interior wall 290 of the compressible member 256 inward and apply pressure radially to the introducer sheath 106.

In general, the compressible member 256 may be made from any material that is suitably compressible to lock the delivery wire within the introducer sheath. Exemplary types of materials include rubbers, synthetic rubbers or other soft polymers, elastomers, and thermoplastic elastomers. Exemplary materials include Santoprenes™, silicones, polyurethanes, polyolefin, fluoropolymer, polyvinylidene fluoride, and Teflon®. The compressible member may be in the form of a deformable structure, which itself may be formed of a deformable material or a material that is not deformable. Exemplary deformable structures include braided, knit or woven metallic or polymeric structures and/or foams. Combinations of materials can be used.

While particular designs of the compressible member 256 have been shown, other designs may also be used. As an example, in some embodiments, the side wall of the compressible member 256 may have only a single taper.

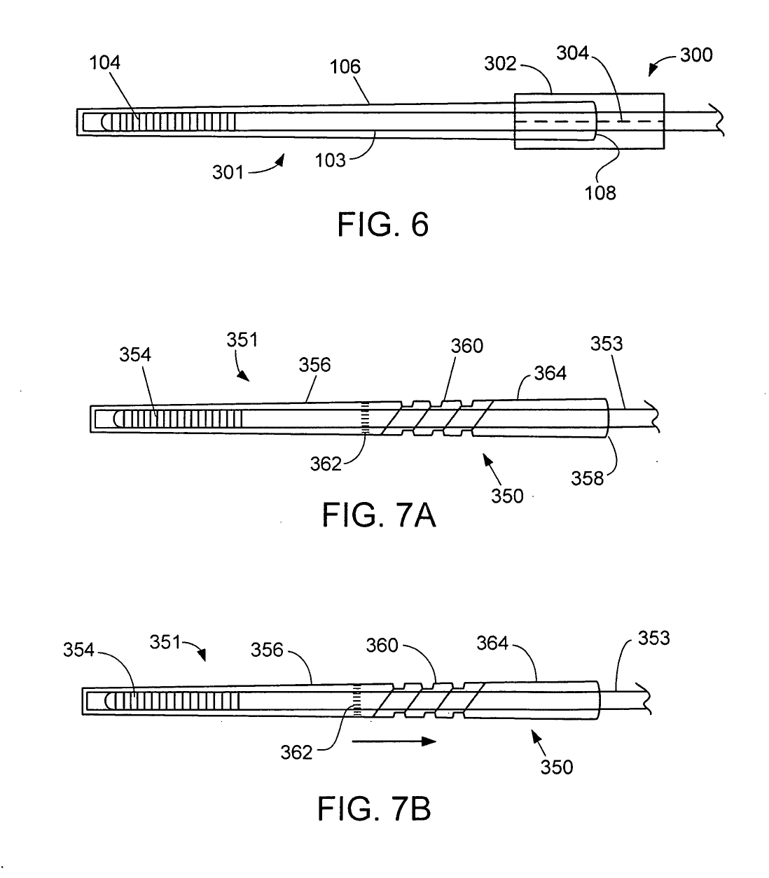

FIG. 6 illustrates another embodiment of an embolic coil introducer system 301, in which the locking mechanism 300 includes a sleeve 302 adapted to fit over the proximal end 108 of the introducer sheath 106 and over a portion of the delivery wire 102 that extends out of the proximal end 108 of the introducer sheath 106. Generally, the fit of the sleeve 302 is sufficiently snug to grip both the introducer sheath 106 and the delivery wire 102 so that sleeve 302 locks the delivery wire 102 in place with respect to the introducer sheath 106. The collar 302 includes a perforation 304 extending axially along the collar 302. Thus, when it is desired to unlock the delivery wire 102, the perforation 304 can be torn and the collar 302 peeled away, allowing delivery wire 102 to move with respect to introducer sheath 106.

Examples of materials from which sleeve 302 can be formed include resilient materials, for example, polymers, such as, for example, rubbers, plastics, and thermoplastic elastomers. In certain embodiments, the sleeve 302 may be formed of a heat shrunk material. Examples of heat shrunk materials include heat shrunk polyvinyl chlorides (PVCs), heat shrunk polytetrafluoroethylenes (PTFEs), heat shrunk polyolefins, heat shrunk fluoroethylene polymers (FEPs), and heat shrunk polyvinylidenedifluorides (PVDFs), and/or polyethylene terephthalates (PETs).

In some embodiments, an elastic sleeve as just described can be employed, where the sleeve has no perforations. The sleeve can be removed, e.g., by rolling the sleeve off of the junction of the introducer sheath and the delivery wire, permitting the delivery wire to be moved with respect to the introducer sheath. The sleeve can, if desired, be returned to the junction of the introducer sheath and the delivery wire, e.g., by rolling the sleeve back onto the junction, to re-lock the delivery wire, such that the delivery wire is substantially immobile longitudinally with respect to the introducer sheath. The sleeve could be formed of any of the resilient materials described above.

FIGS. 7A and 7B show an embodiment of an embolic coil introducer system 351 that includes a locking mechanism 350 that is unitary with an introducer sheath 356. In general, the introducer sheath 356 is formed of a heat-deformable material (e.g., a thermoplastic such as a polypropylene). A twist-lock 360 is located in a proximal portion 364 of the introducer sheath 356. Twist-lock 360 can be formed, for example, by applying heat to a small region of the introducer sheath 356 and twisting the sheath 356 tightly about a delivery wire 353 contained within the sheath so that the twist-lock portion of the sheath 356 becomes engaged around the delivery wire 353. When it is desired to move the delivery wire 353 within the introducer sheath 356, the twist-lock 360 is disengaged by rotating it in the opposite direction to that of the formation of the twist-lock 360. To prohibit the twist-lock 360 from inhibiting the free movement of the delivery wire 353 within the introducer sheath 356, a perforation 362 is included distally of the twist-lock. The introducer sheath 356 can be torn and the proximal portion 364 of the introducer sheath 356 can be removed from the delivery wire. Optionally, sheath 356 may include a perforation that extends longitudinally along the length of the proximal portion of the introducer sheath (e.g., to allow the portion to be removed without necessitating sliding it to the proximal end of the delivery wire). In some embodiments, a perforation is not included in sheath 356.

While certain embodiments have been described, others are possible.

As an example, in some embodiments, the locking mechanism could involve an iris-type opening for applying pressure radially against the introducer sheath.

As another example, while certain embodiments of springs have been described, other embodiments can be used. In some embodiments, the spring may be a leaf spring or a coiled spring. In certain embodiments, the spring may be unitary with the first or second extension. In some embodiments, the first extension, second extension and the spring may all be unitary and the biasing of the first and second extensions may arise from the configuration of these unitary components.

As a further example, while certain locking materials for locking mechanisms have been described, other materials can be used. In some embodiments, the locking mechanism can be formed of a shape memory material (e.g., a shape memory metal, a shape memory polymer). Typically, such materials are shaped to a first shape at a high temperature and are reshaped to a second shape following cooling, and upon subsequent reheating, the materials revert to the first shape.

As yet another example, in certain embodiments the introducer sheath can have an outer diameter of no more than about 2 mm (e.g., no more than about 1.5 mm, no more than about 1 mm, or no more than about 0.7 mm), and/or no less than about 0.5 mm (e.g., no less than about 0.7 mm, no less than about 1 mm, or no less than about 1.5 mm). For example, the introducer sheath can have an outer diameter of between about 0.5 mm and about 2 mm (e.g., between about 0.7 mm and about 1.5 mm).

As an additional example, in certain embodiments, the introducer sheath can have an inner diameter of no more than about 1 mm (e.g., no more than about 0.5 mm, no more than about 0.4 mm, or no more than about 0.3 mm), and/or no less than about 0.2 mm (e.g., no less than about 0.3 mm, no less than about 0.4 mm, or no less than about 0.5 mm). For example, in some embodiments, the introducer sheath can have an inner diameter of between about 1 mm and about 0.2 mm (e.g., between about 0.5 mm and about 0.2 mm, or about 0.4 mm and about 0.3 mm).

Other embodiments are in the claims.

What is claimed is:

1. A system, comprising:
an embolic coil introducer sheath; and
a locking mechanism having a first position in which the locking mechanism applies pressure to the embolic coil introducer sheath and a second position in which the locking mechanism does not apply pressure to the embolic coil introducer sheath, wherein the locking mechanism is removable from the embolic coil introducer sheath.

2. The system of claim 1, wherein the locking mechanism can be moved from the first position to the second position.

3. The system of claim 1, wherein the locking mechanism can be repeatedly moved between the first position and the second position.

4. The system of claim 1, wherein the locking mechanism comprises a clamp having first and second extensions.

5. The system of claim 4, wherein the first and second extensions are unitary with each other.

6. The system of claim 4, wherein the first and second extensions are biased to apply pressure at at least two points substantially opposite each other around the embolic coil introducer sheath.

7. The system of claim 6, wherein the extensions are biased by a spring.

8. The system of claim 6, wherein the first and second extensions are biased toward each other.

9. The system of claim 4, wherein the first and second extensions have sheath-contacting surfaces operative to contact the introducer sheath when the locking mechanism is in the first position, and the sheath-contacting surfaces are configured to at least partially envelope a portion of the introducer sheath.

10. The system of claim 4, wherein the clamp further comprises:
a locking portion extending from the first extension; and
a ratchet extending from the second extension and adapted to engage the locking portion of the first extension,
wherein the first and second extensions are biased away from each other and the locking extension and ratchet, when engaged, operate to hold the clamp in the first position.

11. The system of claim 1, wherein the locking mechanism comprises:
a resilient material having a bore; and
a member configured to translate an axial force on the member into a radial compressive force on the embolic coil assembly.

12. The system of claim 11, wherein the locking mechanism comprises a Touhy-Borst valve.

13. The system of claim 11, wherein the locking mechanism comprises:
a receiving member having a central bore, the receiving member comprising:
a socket segment having threads; and
a tapered portion abutting the socket segment and having a conical internal surface with its largest internal diameter abutting the socket segment;
a cap having a central bore, the cap comprising:
a threaded portion operative to engage the threads of the socket segment of the receiving member; and
a tubular extension extending longitudinally away from the threaded portion and having a generally tapered surface at the end thereof; and
a resilient member having a central bore, the resilient member having a cone-shaped portion with the smallest cross-sectional diameter at an end of the resilient member;
wherein the resilient member resides between the receiving member and the cap such that axial compression of the resilient portion, from threading the cap onto the receiving member, is converted to radial compressive force that biases the resilient material radially inward into the central bore.

14. The system of claim 1, wherein the locking mechanism has a wedge-shaped portion.

15. The system of claim 14, further comprising an embolic coil pusher wire, wherein the locking mechanism is configured to at least partially fit between a sheath and the embolic coil pusher wire.

16. The system of claim 14, wherein the locking mechanism has one or more slots extending from a proximal region of the wedge-shaped portion of the locking mechanism to a distal region of the wedge-shaped portion of the locking mechanism.

17. The system of claim 16, wherein at least one slot extends a full length of the wedge-shaped portion of the locking mechanism.

18. The system of claim 14, wherein the wedge-shaped portion of the locking mechanism includes one or more protrusions at a proximal end of the wedge and extending radially outward.

19. The system of claim 14, wherein the wedge-shaped portion of the locking mechanism comprises a resilient material.

20. The system of claim 19, wherein the resilient material comprises a material selected from the group consisting of rubbers, plastics, foams and combinations thereof.

21. The system of claim 1, wherein the locking mechanism is in the form of a sleeve.

22. The system of claim 21, wherein the sleeve comprises a heat-shrinkable material.

23. The system of claim 22, wherein the heat-shrinkable material is selected from the group consisting of polyvinyl chloride (PVC), polytetrafluoroethylene(PTFE), polyolefin, FEP, polyvinylidenedifluoride (PVDF), and combinations thereof.

24. The system of claim 21, wherein the sleeve is perforated.

25. The system of claim 1, further comprising:
an embolic coil assembly, comprising:
an embolic coil; and
an embolic coil pusher wire,
wherein the embolic coil assembly is at least partially contained within the embolic coil introducer sheath.

26. The system of claim 25, wherein the locking mechanism does not directly contact the embolic coil assembly.

27. The system of claim 25, wherein, when the locking mechanism is in the first position, the locking mechanism applies pressure to the embolic coil assembly through the embolic coil introducer sheath.

28. The system of claim 25, wherein the locking mechanism directly contacts the embolic coil assembly.

29. The system of claim 28, wherein the locking mechanism includes a wedge-shaped portion that is operative to fit between the embolic coil assembly and the embolic coil introducer sheath.

30. The system of claim 29, wherein the wedge-shaped portion of the locking mechanism extends around the full circumference of the embolic coil assembly.

31. The system of claim 29, wherein the wedge-shaped portion has a substantially C-shaped cross section.

32. The system of claim 29, wherein the wedge-shaped portion has a substantially O-shaped cross-section.

33. The system of claim 28, wherein the locking mechanism is in the form of a sleeve, the sleeve being supported by a proximal end of the embolic coil introducer sheath, the sleeve being supported by a portion of the embolic coil assembly that extends from the proximal end of the introducer sheath.

34. The system of claim 33, wherein the sleeve is heat shrunk onto the proximal end of the embolic coil introducer sheath, and the sleeve is heat shrunk onto the portion of the embolic coil assembly that extends from the proximal end of the introducer sheath.

35. The system of claim 33, wherein the sleeve is perforated.

36. A system, comprising:
an embolic coil introducer sheath; and
a locking mechanism having first and second position,
wherein:
the locking mechanism applies pressure to the embolic coil introducer sheath when the locking mechanism is in the first position;
the locking mechanism does not apply pressure to the embolic coil introducer sheath when the locking mechanism is in the second position; and
the locking mechanism can be moved from the second position to the first position.

37. A system, comprising:
an embolic coil introducer sheath; and
a locking mechanism having first and second position,
wherein:
the locking mechanism applies pressure to the embolic coil introducer sheath when the locking mechanism is in the first position;
the locking mechanism does not apply pressure to the embolic coil introducer sheath when the locking mechanism is in the second position; and
the locking mechanism can be repeatedly moved between the first and second positions.

38. A system, comprising:
an embolic coil introducer sheath; and
a clamp having first and second positions,
wherein the clamp applies pressure to the embolic coil introducer sheath when the clamp is in the first position, and the clamp does not apply pressure to the embolic coil introducer sheath when the clamp is in the second position.

39. A system, comprising:
a resilient material having a bore;
an embolic coil introducer sheath disposed in the bore of the resilient material; and
a member configured to translate an axial force on the member into a radial compressive force on the embolic coil introducer sheath.

40. A system, comprising:
an embolic coil introducer sheath; and
a Touhy-Borst valve configured to apply pressure against the embolic coil introducer sheath.

41. A system, comprising:
an embolic coil introducer sheath;
an embolic coil pusher wire; and
a wedge configured to fit between the embolic coil introducer sheath and the embolic coil pusher wire.

42. A system, comprising:
an embolic coil introducer sheath; and
a sleeve comprising a polymer material,
wherein the sleeve is supported by the embolic coil introducer sheath, and the sleeve has at least one perforation.

43. A perforated embolic coil introducer sheath.

44. A system, comprising:
an embolic coil introducer sheath having at least one perforation; and
an embolic coil pusher wire,
wherein the perforated embolic coil introducer sheath comprises a locking portion that restricts longitudinal movement of the embolic coil pusher wire in the perforated embolic coil introducer sheath when the locking portion is in a first position and does not restrict longitudinal movement of the embolic coil pusher wire in the perforated embolic coil introducer sheath when the locking portion is in a second position.

45. The system of claim 44, wherein the at least one perforation of the embolic coil introducer sheath is located distally of the locking portion of the embolic coil introducer sheath.

46. The system of claim 44, wherein the at least one perforation is configured to permit the locking portion to be torn off of the embolic coil introducer sheath.
